(19) United States
(12) Patent Application Publication
(10) Pub. No.: US 2009/0285353 A1
(43) Pub. Date: Nov. 19, 2009

(54) Array CT

(75) Inventors: Michael P. Ellenbogen, Wayland, MA (US); Richard Bijjani, Cambridge, MA (US); Michael Litchfield, Winchester, MA (US); Peter Conway, Pepperell, MA (US); William Aitkenhead, Sharon, MA (US); Bruce Lee, Winchester, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Reveal Imaging Technologies, Inc., Bedford, MA (US)

(21) Appl. No.: 12/468,714

(22) Filed: May 19, 2009

Related U.S. Application Data
(60) Provisional application No. 61/054,411, filed on May 19, 2008.

(51) Int. Cl. A61B 6/00; G01N 23/04

(52) U.S. Cl. 378/9; 378/57

(57) ABSTRACT

Embodiments of an Array CT scanning system for x-ray scanning objects (e.g., scanning airline baggage, packages, and cargo) can include a conveyor configured to transport baggage through a tunnel, a bottom mounted x-ray source configured to provide five fan beams through the tunnel, a side mounted x-ray source disposed at a height higher than the conveyor and configured to provide a fan beam through the tunnel, and a plurality of detectors disposed across the arcs of each of the fan beams. An image processing system can be configured to provide 3D type images of a scanned bag as a function of the information received from the detectors. The images can be derived through interpolation of the scan data. An operator can manipulate the image data and partially rotate the bag to discern objects located within. A side tray is provided to allow an operator to remove a suspect bag from an operational flow of bags. Image information can be stored for subsequent review. Multiple scanners can be networked together such that image and passenger information can be transferred to other workstations.

### CROSS-REFERENCE TO RELATED ACTIONS

This application claims the benefit of U.S. (Provisional) Application No. 61/054,411, filed on May 19, 2008, which is incorporated herein by reference.

### BACKGROUND

Security checkpoints, such as those located in airports, screen people and packages for contraband, such as weapons or explosives. Various technologies are used at such checkpoints. At an airport, passenger baggage typically moves on a conveyor through a projection x-ray system and an operator can review images of screened baggage to determine whether the baggage includes contraband. Operators receive training to recognize certain types of objects in an x-ray image. Furthermore, a typical operator receives training to distinguish objects layered within the bags from a single two dimensional x-ray image. It can be difficult, however, for an operator to distinguish contraband in single view scanners because of occluding and overlapping objects in the image.

Multi-view x-ray systems have been used to provide additional x-ray images of baggage. These systems typically include an x-ray sources placed below and at the side of the inspection tunnel, thus providing two or more orthogonal views of the baggage. These systems, however, still present challenges to the operator (i.e. security screener) due to occluding on overlapping objects. For example, it is often difficult for an operator to determine whether they are looking at a single object or two separated objects that are overlapping in the x-ray image. As a result of the uncertainty in the image, a baggage item may have to be scanned again at a different angle or manually searched, resulting in a loss of time and increase delays for the passengers.

Accordingly, there is a need increase the image quality and detection algorithms in multi-view x-ray scanning systems.

### SUMMARY

In general, in an aspect, the invention provides an x-ray scanning system including a conveyor located at least partially in a tunnel and configured to move an object to be scanned through the tunnel along a direction of travel, a first x-ray source located beneath the tunnel and configured to project one or more fan beams from a first focal point through the tunnel, a first plurality of detector arrays, such that each of the detector arrays is aligned to one of the fan beams projected from the first x-ray source, a second x-ray source located on the side of the tunnel and configured to project a fan beam from second focal point through the tunnel, and a second detector array aligned to the fan beam projected from the second x-ray source.

Implementations of the invention may include one or more of the following features. The second focal point can be a height that is higher than the conveyor. The height of the second focal point can be approximately 8 inches above the conveyor. The first x-ray source can be configured to project five fan beams from the first focal point. The angle between each of the fan beams can be approximately 12.5 degrees. An image processing system can be configured to generate 3D images of a scanned object. An operator station can include a display monitor and an input device, such that an operator at the station can manipulate the input device to rotate the 3D images around a first pivot point. The operator can manipulate the input device to rotate the 3D images around a second pivot point. The first x-ray source can be located in front of the second x-ray source along the direction of travel.

In general, in another aspect, the invention provides an array CT scanning system, including a tunnel, a conveyor located at least partially within the tunnel can configured to move an object to be scanned through the tunnel along a direction of travel, a single detector array located near the tunnel, more than one x-ray sources located on the tunnel along the direction of travel, such that each x-ray source is configured to project a fan beam towards the single detector, and a control system connected to the x-ray sources and configured to activate each of the x-ray sources sequentially such that only one x-ray source is projecting at a time.

Implementations of the invention may include one or more of the following features. The x-ray sources can be located under the tunnel and the fan beams can be projected to the top and a side of the tunnel. The x-ray sources can be located on the side of the tunnel and the fan beams are projected to a side, the top, and the bottom of the tunnel. The x-ray sources can be a single source such as one using nanotube technology and is configured to project the fan beams to the single detector. The single detector array can include more than one detector elements, such that each element can include a low energy detector, a high energy detector, and a curved filter material positioned between the low and high energy detectors. The curved filter material can be located in the detector array such that each of the fan beams generated from each of the x-ray sources is substantially normal to the surface of the curved filter.

In general, in another aspect, the invention provides a passenger baggage screening system, including a multi-beam x-ray scanner with a conveyor, an operator image display screen, a side tray disposed adjacent to the conveyor such that a bag under inspection can be moved from the conveyor to the side tray by an operator, and a bin return system.

Implementations of the invention may include one or more of the following features. An image processing system can be configured to store image information. the stored image information can be selected and displayed on the operator image display screen. The operator image display screen can include an input device. The image information can be 3D images of passenger baggage, and an operator can manipulate the input device to display and rotate the 3D images around a selectable pivot point.

In accordance with implementations of the invention, one or more of the following capabilities may be provided. Passenger baggage can be screened for contraband with improved detection rates as compared to conventional x-ray scanners. Government and security agency requirements for the screening of passenger carry-on items by providing a two-level x-ray screening device with advanced multi-view dual-energy technology can be achieved. 3D-like bag images can be generated an reviewed in real-time. A security officer can rotate high resolution bag images to inspect for potential threat objects and their surroundings. Detection of liquids can be increased. Algorithms to automatically detect threat materials, including liquids and homemade explosives (HMEs) can be implemented. Divest and Revest Stations, System Conveyor, and Bin Return System can improve passenger throughput, and reduce labor costs. Images can be transferred to a Remote Resolution Workstation without stopping the operational flow of bags through the system.

These and other capabilities of the invention, along with the invention itself, will be more fully understood after a review of the following figures, detailed description, and claims.

### BRIEF DESCRIPTION OF THE FIGURES

FIG. 1 is a view into the tunnel of a prior art multi-beam x-ray scanner illustrating a tunnel and three sets of an x-ray source and an L-shaped detector.

FIGS. 2A-2C are perspective, top, and side beam diagrams depicting a tunnel with a bottom mounted x-ray source collimated into five wide angle beams, and a side mounted x-ray source collimated into a single wide angle beam.

FIG. 3 is a view into the tunnel illustrating a wide angle beam radiating from a bottom mounted x-ray and corresponding detector arrays.

FIG. 4 is a view into the tunnel of a wide angle beam radiated from the side mounted x-ray and corresponding detector arrays.

FIG. 5 is a perspective view of a scanner assembly including a tunnel and a plurality of detector arrays.

FIG. 6 is a perspective view of an exemplary an x-ray detector element.

FIG. 7 is a perspective view of a multi-beam x-ray scanner and baggage handling assembly, including a screenshot of a graphic user interface including an x-ray image.

FIG. 8 is a series of images depicting the rotation of a bag around a first rotation axis.

FIG. 9 is a series of images depicting the rotation of a bag around a second rotation axis.

FIG. 10 is a graph of an image constructed from the side mounted x-ray source and associated detectors.

FIGS. 11A-C are a collection of block diagrams depicting configurations for bottom mounted x-ray sources and detector assemblies.

FIG. 12A-C are a collection of block diagrams depicting configurations for side mounted x-ray sources and detector assemblies.

FIG. 13 is a block diagram depicting a multi-source and multi-detector scanning system.

FIGS. 14 and 15 are block diagrams of detector elements for use in multi-source single detector array configuration.

FIG. 16 is a flow chart for determining the Zeff of an object.

FIGS. 17A-17D are conceptual diagrams associated with the Zeff calculation.

FIGS. 18A-B includes block diagrams for container inspection embodiments of an Array CT scanner.

### DETAILED DESCRIPTION OF PREFERRED EMBODIMENTS

Embodiments of the invention provide techniques for x-ray scanning objects (e.g., scanning airline checked or carry-on baggage for contraband). For example, an Array CT scanner system includes a conveyor configured to transport baggage through a tunnel, a bottom mounted x-ray source configured to provide five fan beams through the tunnel, a side mounted x-ray source disposed at a height higher than the conveyor and configured to provide a fan beam through the tunnel, and a plurality of detectors disposed across the arcs of each of the fan beams. The scanner includes an image processing system configured to provide 3D type images of a scanned bag as a function of the information received from the detectors. An operator can manipulate the image and partially rotate the bag to discern objects located within. A side tray can be provided to allow an operator to remove a suspect bag from an operational flow of bags. Image information can be stored for subsequent review. Multiple scanners can be networked together such that image and passenger information can be transferred to other workstations. This scanner is exemplary, however, and not limiting of the invention as other implementations in accordance with the disclosure are possible.

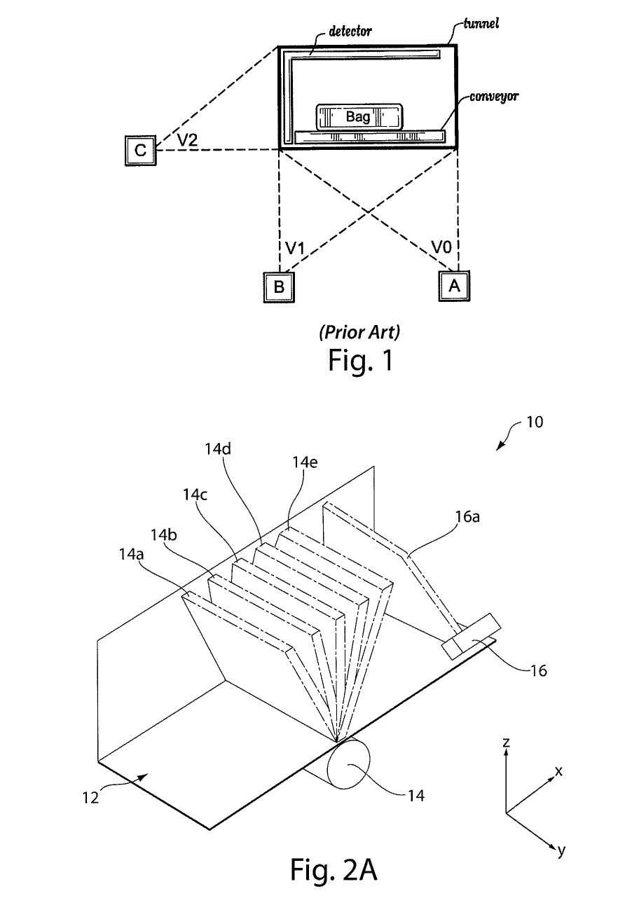

Referring to FIG. 1, is a tunnel view of a prior art scanner with multiple x-ray sources is shown. The system includes x-ray sources A, B, and C, that produce respective fan-shaped x-ray beams V0, V1, and V2. Each of these beams conforms to a respective plane, and the three planes that are parallel to each other are spaced from each other in the direction of movement of the bag. Accordingly, since this prior art system is capable of providing only images based on orthogonal views of the bag, in can be difficult for an operator and/or an automated detection algorithm to discriminate objects in these images due to their orientation as well as occluding and overlapping objects in the images.

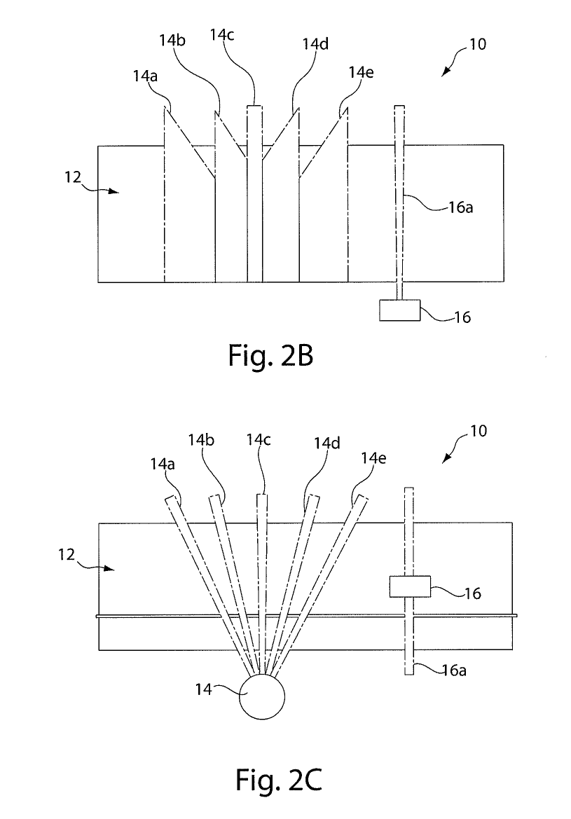

Referring to FIGS. 2A-2C, an Array CT scanner 10 includes a tunnel 12, a bottom x-ray source 14, a side x-ray source 16, and a plurality of dual-energy detector arrays (not shown). The Array CT scanner utilizes Kinetic Depth X-ray Imaging (see J. P. O. Evans, J. W. Chan, V. Vassiliades, and D. Downes, “Kinetic Depth X-ray (KDEX) Imaging for Security Screening,” The 4th International Aviation Security Technology Symposium 2006, and J. P. O. Evans, “Kinetic depth effect X-ray (KDEX) Imaging for Security Screening,” The International Conference on Visual Engineering, 2003). Both of these references are incorporated by reference. In an embodiment, the scanner can include a wide-angle cone x-ray source 14 placed below the tunnel 12. In general, the cone x-ray source can be collimated into n number of fan beams (e.g., 1, 2, 3, 4, 5, 6, 7, 8) as required for the scanning application. In an embodiment, the bottom mounted x-ray source 14 can be configured to provide five fan beams 14a-e. In this embodiment, a set of five dual-energy detector arrays can be disposed around portions of the tunnel 12, and can be configured to intercept the fan beams 14a-e. As an example, and not a limitation, the detector arrays can be spread out in a fan formation from the x-ray source 14 with approximately 12.5 degrees of separation. Different configurations, using a different number of detector arrays which are disposed at different angles can be used. The scanner also can include a wide-angle x-ray source 16 placed at the side of the tunnel 12 and can be configured to produce a fan beam 16a extending across the tunnel 12. The side x-ray source 16 can be mounted in a location that is higher than the bottom of the tunnel 12 to provide a more direct view of a liquid surface, such as a water bottle or a soda can in a carry-on bin. In general, the height of the side x-ray source 16 can be approximately the level of the top of a carry-on bin (e.g., 8, 10, 12 inches). In general, the elevation of the side x-ray source 16 increases chance of imaging the top of the liquid level in container. In operation, the tunnel 12 can include a conveyor to move an object (e.g., baggage) through the tunnel 12 and the fan beams (i.e., 14a-e, 16a). The rate of travel for the conveyor can be adjusted or reversed based on the needs of an operator, and/or an associated image processing system.

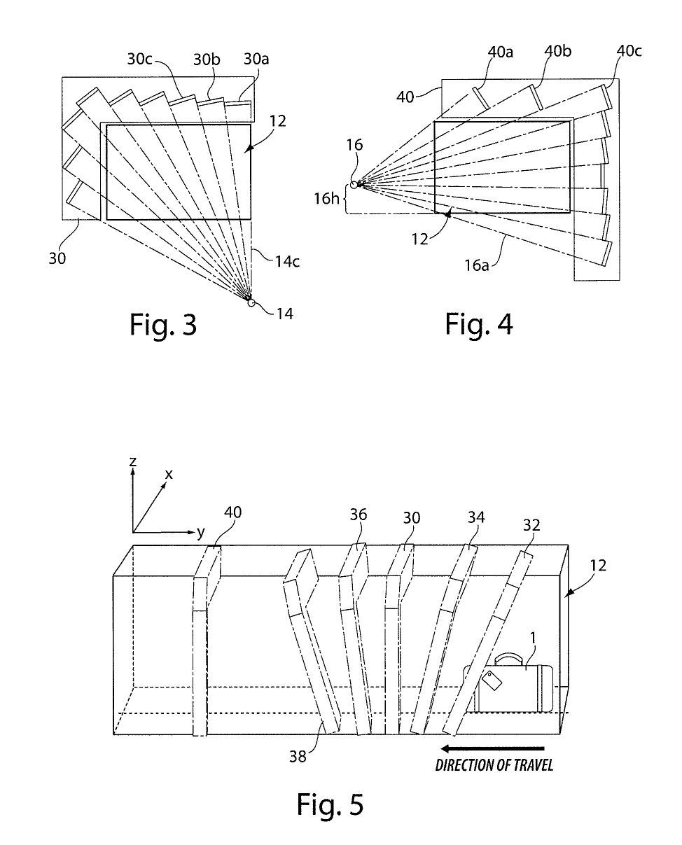

Referring to FIG. 3, with further reference to FIGS. 2A-2C, an exemplary dual-energy detector array 30 for the bottom mounted x-ray source 14 is shown. The detector array 30 includes a plurality of detector elements (e.g., 30a, 30b, 30c) disposed around the tunnel 12 such that the plurality of detectors elements intercept a significant portion of the wide-angle x-ray beams (e.g., the center beam 14c). The center of each detector element (e.g., 30a, 30b, 30c) is mounted on the array 30 such that the center is approximately orthogonal to the x-ray source 14. The number and size of the detector elements (e.g. 30a, 30b, 30c) is exemplary only, and can change based on required performance parameters and materials used (e.g., x-ray source, detector material, baffles, beam guides). The detectors (e.g., 30a, 30b, 30c) can be positioned so that the end of the detector is substantially adjacent to detectors on either side of it. Ideally, for purposes of reconstruction, every detector in the array would be perpendicular to and equidistant from the x-ray source. In operation, objects to be inspected (e.g., baggage, packages, cargo) can be disposed within the fan beams (e.g., 14c, 16a) between the x-ray source and the detectors arrays. The detector arrays can be configured to transmit detection information to an image processing computer. The image processing computer can include a processor, memory and computer readable instructions on a computer readable medium, and is configured to transform the detection information into image information. For example, the mass, location and density of objects in the baggage can be determined.

Referring to FIG. 4, with further reference to FIGS. 2A-2C, an exemplary dual-energy detector array 40 for the side mounted x-ray source 16 is shown. The detector array 40 can include a plurality of detectors elements (e.g., 40a, 40b, 40c) disposed around the tunnel 12 such that the plurality of detectors intercept a significant portion the wide-angle x-ray beam 16a. The center of each detector element (e.g., 40a, 40b, 40c) can be mounted on the array 40 such that the center is approximately orthogonal to the x-ray source 16. The number and size of the detector elements (e.g. 40a, 40b, 40c) is exemplary only, and can change based on required performance parameters and materials used (e.g., x-ray source, detector material, baffles, beam guides). The side mounted x-ray source 16 can be disposed at a height 16h which based on performance factors such as the dimensions the tunnel 12, and/or of the carry-on bins used to convey objects through the tunnel 12. The height is exemplary only, and can be modified based on the dimensions of the tunnel 12.

Referring to FIG. 5, with further reference to FIGS. 3 and 4, a perspective view of a scanner assembly 50 is shown. The scanner 50 includes a tunnel 12, a bottom mounted wide array x-ray source (not shown) with a plurality of detector arrays 30, 32, 34, 36, 38, and a side mounted x-ray source (not shown) with detector array 40. The scanner 50 includes other items that are not shown. In one embodiment, the detector arrays 30, 32, 34, 36, 38 can be spread out in a fan formation from the bottom mounted x-ray. The number of detection arrays is exemplary and not limiting as a different number of detector arrays can be used (e.g., 2, 3, 4, 6, 7, 8). The tunnel 12, x-ray sources 14, 16, and corresponding detector arrays 30, 32, 34, 36, 38, 40 can be sized based on the items to be scanned. For example, a tunnel dimension of 60 cm×40 cm can be used for screen passenger carry-on baggage in a terminal, a 75 cm×55 cm can be used to inspect passenger's checked baggage 1, and a 1 m×1.8 m tunnel can be used to inspect cargo. In operation, as previously described, an item to be inspected (e.g., baggage 1) is transported down the tunnel 12 via a conveyor system. The baggage 1 is disposed between the x-ray sources (i.e., the bottom source and the side mounted source) and the detector arrays 30, 32, 34, 36, 38, 40.

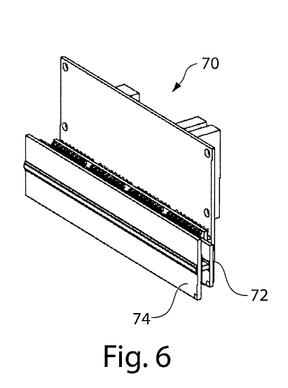

According to an embodiment, the scanner 50 operates in a dual energy mode. Referring to FIG. 6, a cross sectional view of a detector element 70 for dual energy operation is shown. The detector element 70 includes a high energy scintillator layer 72, and a low energy scintillator layer 74. The detector elements can also be configured with collimator material such as collimating plates or a bucky grid to reduce scatter and increase the signal-to-noise ratio of the received x-ray energy. Alternatively, a dual energy scan can be performed using known techniques with a pulsing x-ray source and a single photodiode layer in the detectors.

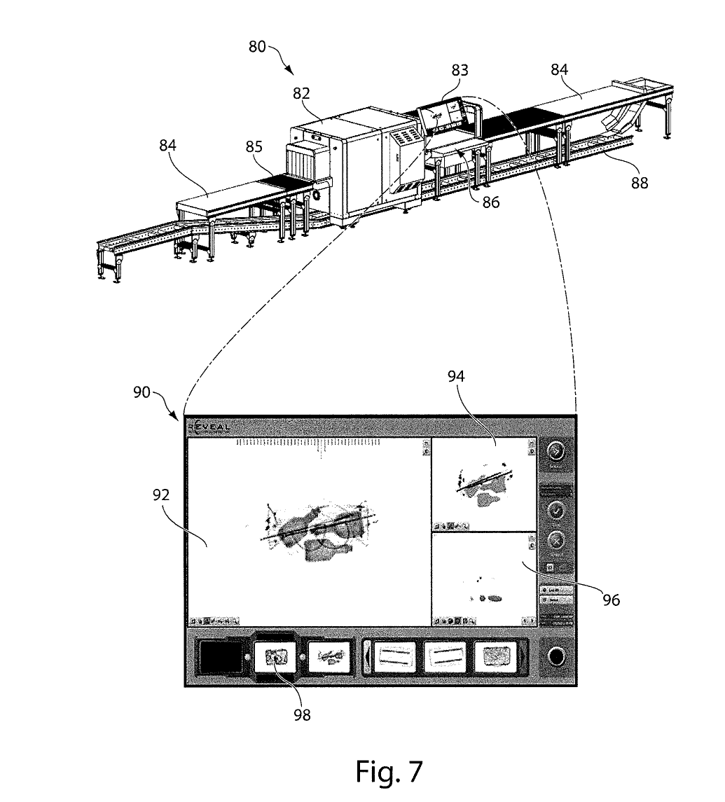

Referring to FIG. 7, with further reference to FIG. 5, a scanner and baggage handling assembly 80 includes a multi-beam scanner 82, an operator image display screen 83, baggage handling tables 84, a conveyor 85, a side tray 86, and a bin return system 88. The scanner 82 includes other items that are not shown. The scanner 82 includes an image processing computer operably coupled to detection arrays within the assembly. In an embodiment, the scanner 82 includes a bottom mounted x-ray source, a side mounted x-ray source and associated detector arrays as previously described in FIGS. 2-6. Other scanner configurations can include additional detectors arrays and x-ray sources, as well as different collimation patterns (e.g., 2, 3, 4, 6, 7, 8 fan beams). The image display screen 83 is operably connected to the image processing computer and is configured to provide image information to an operator via at least one algorithm or program. For example, the image display can be a touch screen LCD configured to display information and receive input from the operator. In general, the scanner 82 includes computers (e.g., control systems, imager processing systems) with processors, memory, operating systems, input and output devices as known in the art. For example, the computers can be multiple computers and/or servers based on Intel® and Motorola® processing structures, and can execute Microsoft Windows®, Linux, and/or Sun® operating systems. The computers can be configured interpret instructions via a computer-readable medium such as floppy disks, conventional hard disks, CD-ROMS, DVDs, Flash ROMS, nonvolatile ROM, and RAM. The computers can be configured to generate and store baggage image and passenger information, as well as transmit and receive such information over a computer network.

In operation, a passenger can place baggage or other items to be scanned (e.g., a bin with personal items such as a laptop or container of liquids) on the table 84. In an embodiment, the scanner installation 80 includes a bin return system 88 to provide a flow of bins to the passengers. The baggage or items can be moved through the scanner 82 via the conveyor 85. The speed and direction of the conveyor can be controlled by the control system computer, and/or the operator. As the baggage moves through the scanner 82, the image processing computer receives scan information from the detectors arrays 30, 32, 34, 36, 38, 40 and computes an image to be displayed on the operation station 83. The operator station 83 can include a screen with a GUI 90. The operator can interactively view the image information through an input device at the operator station 83 (e.g., via the touch screen, joystick, keyboard). For example, to better view objects that are occluded within the bag, the operator can rotate the image 92 through approximately 50 degrees along one axis. The operator can also change the pivot point of the location to better discern two or more objects in the baggage. The extent of the rotation is exemplary and not a limitation as the amount of rotation can increase or decrease as a function of the x-ray source and detector array configuration. A side view of the bag 94 can also be presented on the GUI 90. Other image processing algorithms can be presented, such a high contrast image 96.

In a typical security checkpoint (e.g., airport security screening), there is a screener reviewing images and a “floater” who manually searches any bags that the screener rejects after a visual review of the x-ray image information. In general, in the prior art, when a screener sees something in an image that may be contraband (e.g., weapons, explosives, controlled substances), they will stop the conveyor and request a bag check from the floater. Often, the screener must wait for the floater to become available, and then must take the time to describe the image information when the floater arrives to the operator station 83. During this period, a prior art system would be idle thus creating delays and increased wait times for the passengers. The scanning system 80, however, overcomes this limitation through the use of the side tray 86 and the operator review screen 83. In operation, the operator can identify a suspect bag based on image information. Rather than halting further scanning, the operator can store the image information and pull the suspect bag from the conveyor 85 to “park” the bag on the side tray 86 while waiting for the floater to assist. During this time, the scanner 82 can continue to scan bags, and the operator can continue to review the associated image information. When the floater arrives to inspect the suspect bag, the operator can select the image information from an inspection history bar 98 to display the image information associated with the parked bag. The ability to continue scanning new bags while a previously scanned bag is parked can save time, increase customer satisfaction, and provide safety efficiencies that are not available on a prior art system.

In an embodiment, the scanner 82 is one of several scanners in a network. The network can include stand alone review stations (i.e., not attached to a scanner and located in a remote location) for additional reviews. Continuing the example above, the floater could access the image and passenger information associated with the suspect bag from the stand alone review station. A clear or hold signal could be sent to the operator to indicate whether a subsequent inspection of the bag is required.

In an embodiment, the scanner 82 can include a Host subsystem including a computer and software for controlling machine operations, acquiring detector data, and providing a graphical user interface to the operator. The Host software can also interface with a remote computer in support of the Field Data Reporting System (FDRS), Threat Image Projection (TIP), OJT, OQT and the Security Technology Integrated Program (STIP). In an embodiment, the FDRS can reside on a separate dedicated computer. The “FDRS computer” can support TIP, OJT, OQT and STIP V3.1. For example, the FDRS computer can direct STIP activities, and can send TIP/OJT/OQT images to the Host. This type of distributed computing architecture can provide several advantages, such as isolating and buffering all disk accesses, TIP image downloads, and STIP interfaces are from the active Host software and algorithm program. In addition, a single FDRS can support multiple scanners 82, creating a single workstation for all data collection and supervisory functions. In general, the FDRS computer can provide hardware to support TIP and STIP. For example, a dedicated 10/100/1000 Base-T Ethernet port is available on the FDRS computer specifically for STIP Agent communication with a TSA STIP Server. The Host software can acquire data in support of these applications in real-time via TCP/IP protocol.

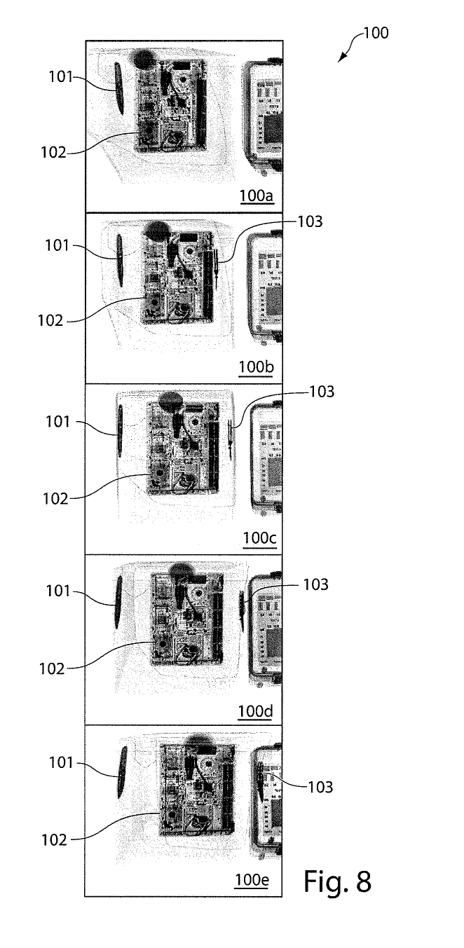

Referring to FIG. 8, with further reference to FIG. 7, a series of images of a bag 100 including a box cutter 101, laptop computer 102, and knife 103 is shown. The images 100a-e are exemplary as more image frames can be generated, and higher frame capture rates can be used. For example, object positions can be determined and displayed through interpolation of the image information (e.g., object-based, Zeff data, high contrast or metal components). An operator can manipulate the workstation 83 to view the image data 100a-e in a rocking motion. That is, the workstation 83 is configured to display the images 100a-e in a flip book manner around a variable pivot point. Accordingly, when the images are viewed in rapid succession, the relative movement of objects in the bag will attract the operator's attention. For example, the bag in this set of images 100 is rotating around a pivot point which is close to the conveyor—i.e. the at the same approximate level as the box cutter 101. In the first image 100a, the laptop 102 is obstructing the view of a knife. In the second image 100b, the knife 103 becomes visible, yet the box cutter 101 does not move appreciably from its location. As the image continues to rotate (i.e., 100c-e), the displacement of the knife 103 increases while the box cutter 101 remains relatively stationary.

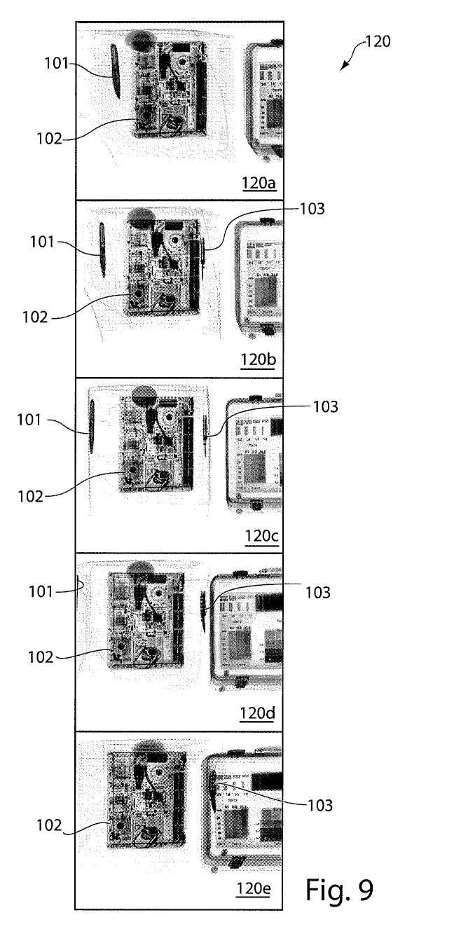

FIG. 9 is another example using the same image information, but with a different pivot point. The images 120 include the same box cutter 101, laptop 102 and knife 103. In this example, the operator has selected a higher pivot point (i.e., a pivot point that is closer to the knife 103). In the first image 120a, the knife is occluded with the laptop 102. As the image is rocked, the second image 120b reveals the knife 103. Since the pivot point is higher, the image of the box cutter 101 is displaced at a greater rate than in the previous example. In the third image 120c, the image of the knife 103 appears to be relatively stationary as compared to the movement of the box cutter image 101. The displacement difference continues in the remaining images 120d-e. In an embodiment, the location of the pivot can be determined automatically by the image processing computers. For example, as noted in the images 100, 120, the laptop 102 can obstruct items located above or below it. The threat detection algorithms in the scanner 82 can identify a laptop in a bag, and select that location as the pivot point. An operator can also manually select or adjust the pivot point during review. The pivot point need not be fixed—the image data can be analyzed with a variety of pivot points in an effort to improve automatic threat detection and operator accuracy.

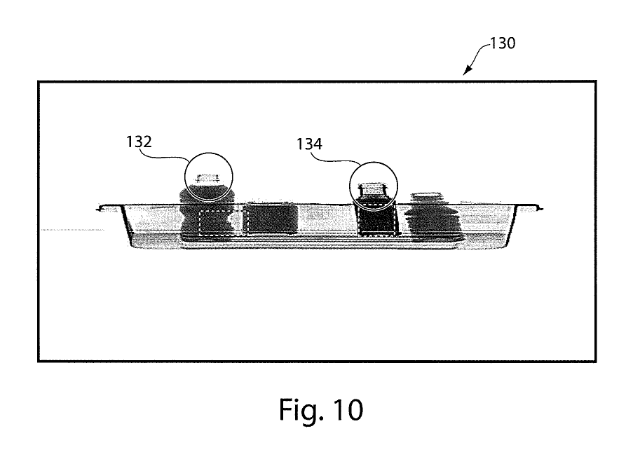

Referring to FIG. 10, with further reference to FIG. 4, a graph of an image 130 constructed from the side mounted x-ray source and associated detectors is shown. The image 130 includes a plurality of liquid containers. The side mounted x-ray source is elevated provides improved image data for resolving the air-liquid interface in a container. This distinction is highlighted on the image 130 via the two circles 132 and 134. In this example, a liquid-air interface in a water bottle 132 and a bottle of oil 134 are easily distinguishable. In systems with a side-shooter x-ray that is mounted in a lower position, the air-liquid interface can be obscured. A distinguishable air-liquid interface can be a significant factor in threat detection.

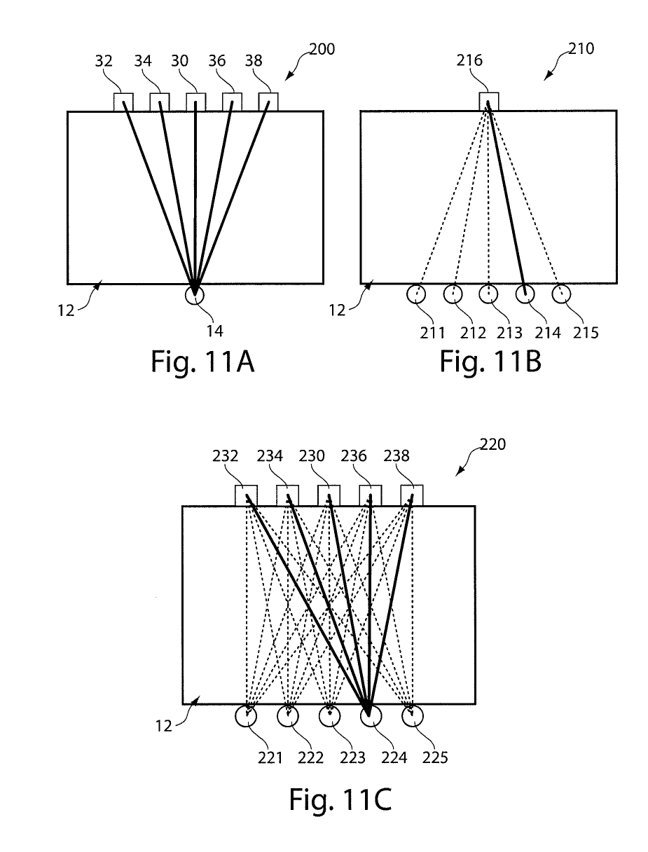

Referring to FIGS. 11A-C, with further reference to FIG. 5, side views of a tunnel 12 with various configurations 200, 210, 220 for side mounted sources and detector assemblies are shown. In an embodiment, as described above, a scanner can include a single source—multi-detector array configuration (FIG. 11A, 200), including an x-ray source 14 and a plurality of detector arrays 30, 32, 34, 36, 38. The source 14 can be a cone beam source which can be collimated into a plurality of fan beams. For example, cone beam x-ray sources can be purchased from commercial sources (e.g., Kaiser Systems, Spellman High Voltage, Comet, Varian, Lohmann). In an alternative embodiment, a scanner can include a multi-source—single detector array configuration (FIG. 11B, 210). Rather than provide a cone beam source 14 to illuminate a large portion of the tunnel 12, the beam path can be reversed such that a single detector 216 can receive energy from multiple x-ray sources 211, 212, 213, 214, 215. In an embodiment, the multi-xray sources could be based on nano-tube technology such as those supplied by Xinray, Nasa Ames, or Thales. Other technologies, such as gridded x-ray sources, which allow fast triggering of the x-ray sources can be used. The x-ray sources 211, 212, 213, 214, 215 can be operably connected to a control system and triggered in sequence such that only one source is active at a time. For example, the configuration 210 indicates that one source 214 is active (i.e. solid line), and the other sources 211, 212, 213, 215 are not active. The multi-source configuration 210 can help reduce the volume of the tunnel 12 that is actively illuminated by x-ray protons from several planes to just one. As a result, the scatter can be reduced and the image resolution can increase. This is particularly relevant when scattering objects such as liquids are in the illumination path.

In an embodiment, referring to FIG. 11C, a scanner can include a multi-source—multi-detector configuration 220. A plurality of x-ray sources 221, 222, 223, 224, 225 can be disposed below the tunnel 12, and a plurality of detector arrays 232, 325, 230, 236, 238 can be disposed along the appropriate parameter. The x-ray sources 221, 222, 223, 224, 225 can provide a wide cone beam which is collimated and directed to each of the detector arrays 232, 325, 230, 236, 238. The x-ray sources can be triggered sequentially. The configuration 220 looses the advantage of reduced scatter as compared to other embodiments (i.e., configuration 210), but can increase the granularity of the angles between views, and can increase the range of angles. As a result, the image processing system can produce smoother active motion between views and can allow an operator, and threat detection algorithms, to see an object with more look angles.

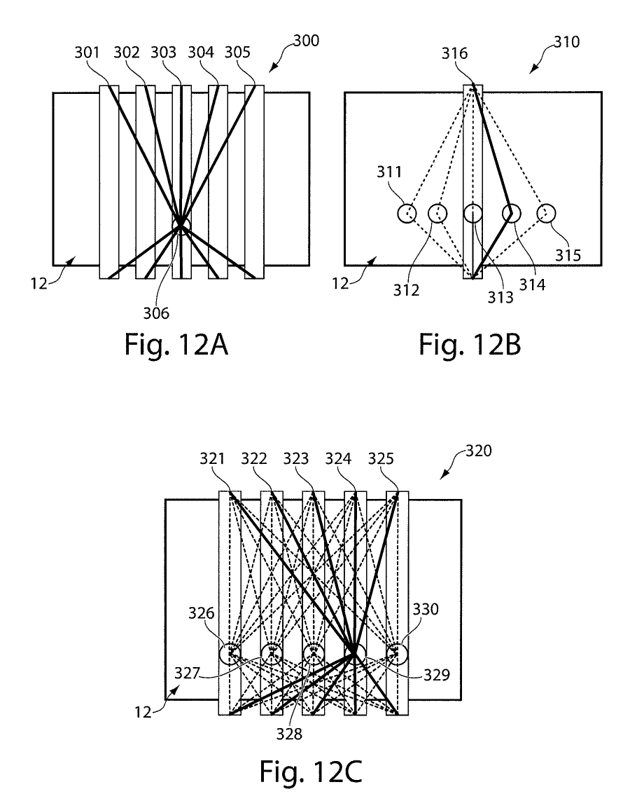

Referring to FIGS. 12A-C, with further reference to FIG. 5, a collection of block diagrams depicting configurations 300, 310, 320 for side mounted x-ray sources and detector assemblies are shown. In an embodiment, the scanner includes a one source—multi-detector array configuration (FIG. 12A, 300). The configuration 300 includes a tunnel 12, a side mounted x-ray source 306, and a plurality of detector arrays 301, 302, 303, 304, 305. The x-ray source 306 is disposed at the side of the tunnel 12, and at a height above the bottom of the tunnel. The x-ray source 306 provides a cone beam, which can be collimated to align with the plurality of detectors 301, 302, 303, 304, 305. As with the bottom mounted x-ray configurations, the multi-detectors enable multi-angle views along the side axis.

In an embodiment, referring to FIG. 12B, the scanner can include a multi-source—single detector array configuration 310. Rather than provide a cone beam source 306 to illuminate a large portion of the tunnel 12, the beam path can be reversed such that a single detector 316 can receive energy from multiple x-ray sources 311, 312, 313, 314, 315. As described above, the multi-xray sources could be based on nano-tube technology, or other technologies which allow fast triggering of the x-ray sources can be used. The x-ray sources 311, 312, 313, 314, 315 can be operably connected to a control system and triggered in sequence such that only one source is active at a time. For example, the configuration 310 indicates that one source 314 is active (i.e. solid line), and the other sources 311, 312, 313, 315 are not.

In an embodiment, referring to FIG. 12C, a scanner can include a multi-source—multi-detector configuration 320. A plurality of x-ray sources 326, 327, 328, 329, 330 can be disposed on the side of the tunnel 12, and a plurality of detector arrays 321, 322, 323, 324, 325 can be disposed along the appropriate parameter. The x-ray sources 326, 327, 328, 329, 330 can provide a wide cone beam which is collimated and directed to each of the detector arrays 321, 322, 323, 324, 325. The x-ray sources 326, 327, 328, 329, 330 can be triggered sequentially. The configuration 320 looses the advantage of reduced scatter as compared to other embodiments (i.e., configuration 310), but can increase the granularity of the angles between views, and can increase the range of angles. As a result, the image processing system can produce smoother active motion between views and can allow an operator, and threat detection algorithms, to see an object with more look angles.

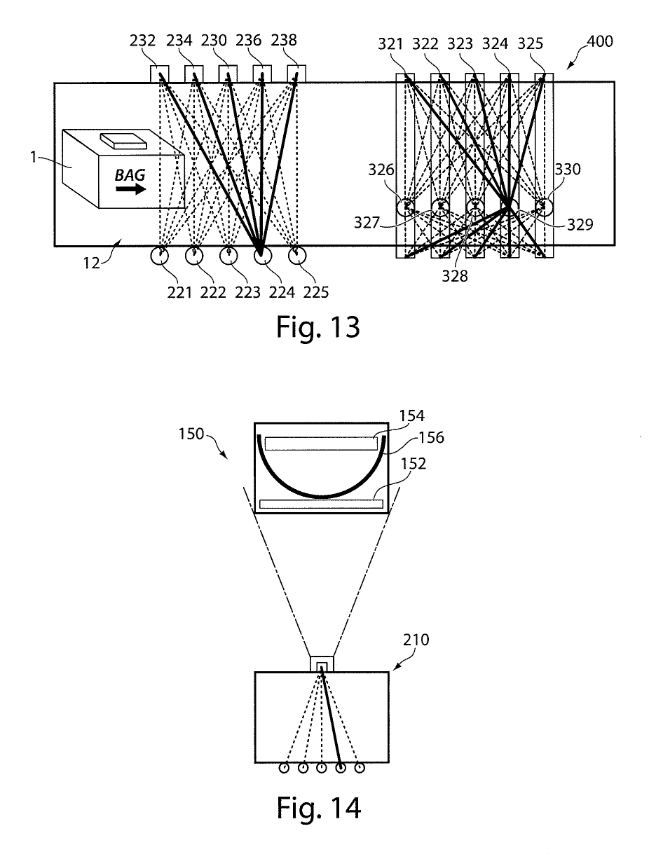

Referring to FIG. 13, with further reference to FIGS. 12 and 13, a block diagram of a multi-source and multi-detector scanning system 400 is shown. The scanner includes a tunnel 12, a plurality of x-ray sources 221, 222, 223, 224, 225 mounted below the tunnel 12, a plurality of bottom-beam detector arrays 230, 232, 234, 236, 238, a plurality of x-ray sources 326, 327, 328, 329, 330 mounted on the side of the tunnel 12 and above the bottom of the tunnel 12, and a plurality of side-beam detector arrays 321, 322, 323, 324, 325. A bag 1 can be placed on a conveyor and moved through the tunnel 12, and through the x-ray beams generated from the x-ray sources 221, 222, 223, 224, 225, 326, 327, 328, 329, 330. The tunnel 12 can include shielding to reduce the amount of x-ray energy escaping from the scanner. The bottom mounted sources 221, 222, 223, 224, 225 can be operably connected to a control system (e.g., processor), and configured to activate sequentially. The corresponding detector arrays 230, 232, 234, 236, 238 receive x-ray energy, and provide image information to an image processing system. The side mounted x-ray sources 326, 327, 328, 329, 330 can be operably connected to the control system, and configured to activate sequentially. The corresponding detector arrays 321, 322, 323, 324, 325 receive x-ray energy, and provide image information to the image processing system. A operator's station, including a computer display and a user interface, can be configured to display image information generated by the image processing system. The image information can include, but is not limited to, rotational views of the bag 1 in at least two axes.

Other combination of source and detector configures can be used. For example, in a cross-over configuration a plurality of x-ray sources can be disposed on the opposite sides of a tunnel and aligned to corresponding detector arrays, which are also on opposing sides. In an embodiment, the locations of the source and detectors cause the corresponding fan beams to cross in the tunnel.

In the embodiment of the invention having multiple radiation sources illuminating a single detector array, the detector can receive radiation from several different angles. In this configuration one detector can be arranged normal to the radiation source and one or more detectors will be arranged off-axis (away from normal) and therefore the detector will detect more photons from the normal radiation source than the other off-axis radiation sources possibly resulting in errors. In this configuration, dual energy detectors which are composed of a low energy detector and a high energy detector separated by a filter (such as brass) need compensation or correction because of the different geometries of the off-axis detectors. For example, the effective thickness of the filter is greater for off-axis radiation sources than the normal radiation source because the off-axis radiation intersects the filter at an angle.

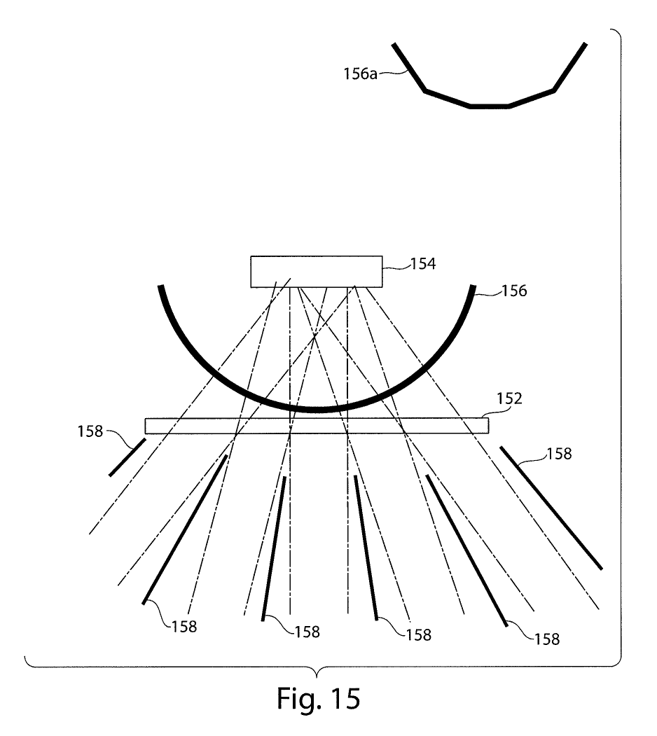

Referring to FIGS. 14 and 15, a detector 150, according to an embodiment, can include a low energy detector 152, a high energy detector 154 and filter material 156. In this embodiment, the filter material 156 can be curved and arranged with the respect to the high energy detector 154 such that the radiation generated by each source is substantially normal to the surface of the curved filter material. In this embodiment, the low energy detector 152 can be larger than the high energy detector 154 in order to extend across the path of the beam produced by each radiation source. The detector 150 can also include one or more collimators 158 arranged between the detector 150 and the radiation source to control the thickness of the beam and ensure that the effective area that is received by each of the detectors 152, 154 is the substantially the same.

The size of the low energy detector 152 and the high energy detector 154 can be determined based on the desired thickness of each of the radiation beams and angles of the off-axis beams with respect to normal. In addition, the radius of curvature of the filter 156 can be selected such that each beam is substantially normal to the surface of the filter 156. The thickness of the beam and the angular orientation of the multiple radiation sources can vary based on the performance requirements of the system. While in the illustrative embodiment, the filter 156 is provided with a curved shape, in alternative embodiments, the filter 156 can be formed in a sequence of flat surfaces 156a, each arranged substantially normal to one of the corresponding beams.

In one embodiment, the system can include five radiation sources arranged at 12.5 degree increments (−25, −12.5, 0, 12.5, 25) which span 50 degrees. The collimators can be arranged to provide a desired beam thickness. The filter, preferably made from a brass material can be curved, or otherwise shaped, as required It should be noted that while the invention is disclosed with respect to a circular filter, other non-circular shapes can be used. For example, the filter can be curved in an elliptical form whereby the beams intersect the filter in a substantially normal direction to the surface of the filter. In another embodiment, the filter can be formed in a sequence of flat surfaces, each arranged substantially normal to one of the corresponding beams.

In operation, each radiation source is energized in a predefined sequence causing a beam to reach the detector at one of the defined angles. The collimator provides that each of the beams substantially uniformly extends over the same area of the detector. The filter can be arranged either in a curved configuration or a set of flat surfaces such that the effective thickness of the filter is substantially the same for each of the beams and the attenuation of each beam by the filter is substantially the same. After passing through the filter, each beam extends over substantially the same area of the high energy detector. As a result, little or no compensation need be applied to each of the signals produced by the detectors from each beam.

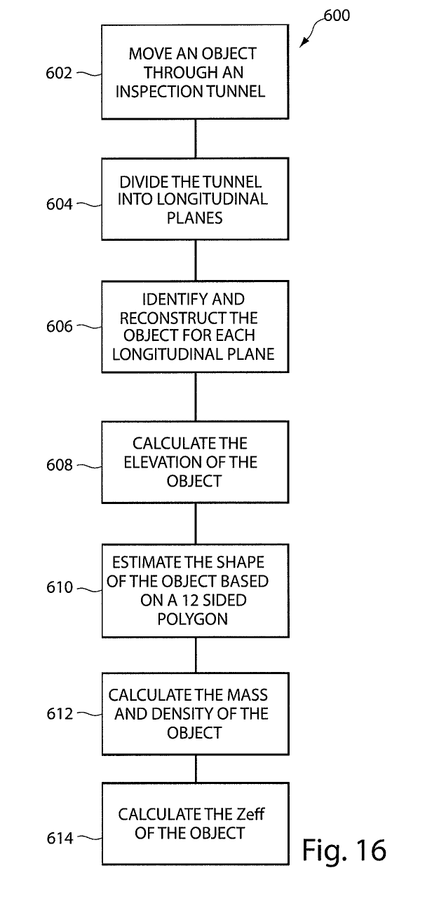

In operation, referring to FIG. 16, with further reference to FIG. 7, a process 600 for—calculating the Zeff of an object using the scanning system 80 includes the stages shown. The process 600, however, is exemplary only and not limiting. The process 600 may be altered, e.g., by having stages added, removed, or rearranged.

Iterative reconstruction techniques are known for CT reconstruction and well defined system solutions such as ART and SIRT. These prior solutions, however, are based on collections of voxels. In contrast, the process 600 reconstructs images a collection of objects of finite sizes and properties.

At stage 602, an object (e.g., baggage, package, container) is moved through an inspection tunnel 12 via a conveyor system. The rate and direction of the movement can be controlled by a control system, which can be operably connected to an image processing system. In an embodiment, the conveyor system includes a single belt with a belt speed of approximately 25 cm per second. For example, given an average bag length of 80 cm and a 20 cm gap between bags, the throughput of the scanning system 80 is approximately 900 bags per hour. Actual throughput in an airport checkpoint, however, can depend on how frequently an operator stops the conveyor belt during operations.

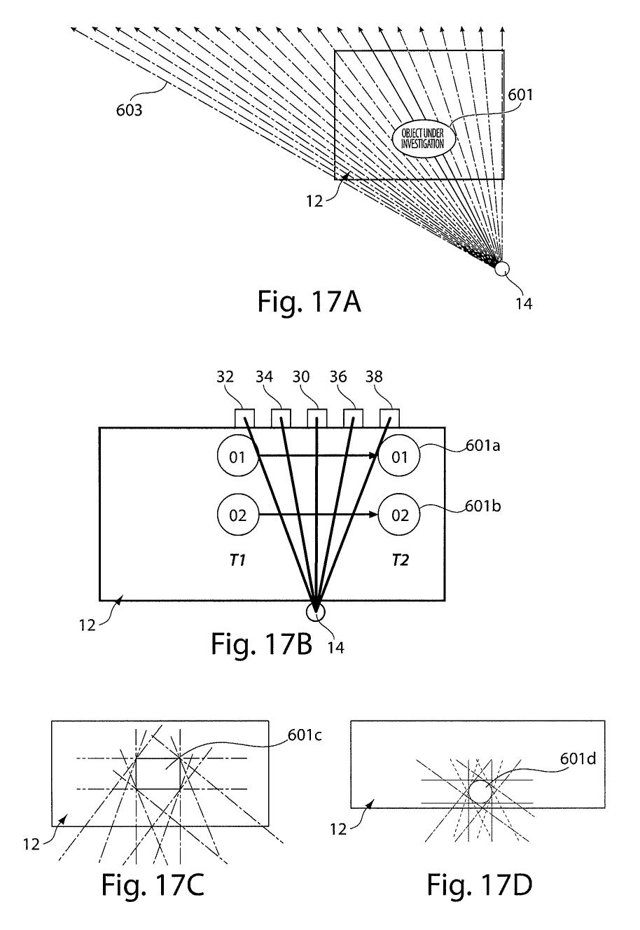

At stage 604, the volume of the tunnel can be analytically divided into longitudinal planes. Referring to FIG. 17A, the bottom mounted x-ray source 14 and corresponding detector arrays functionally divides the tunnel 12 into longitudinal planes 603. Each of the planes 603 is analyzed separately to determine where an object of interest 601 lies. Looking into the tunnel 12, the planes 603 are mapped such that the same number of detectors from different views for a straight line when connected together, and form a unique plane when connected to a focal spot. The tunnel 14 can be divided into hundreds of reconstruction planes. For example, the system 80 includes 780 planes, but the number of planes can be adjusted based on the expected sizes of the objects under investigation. At stage 606, in each of the longitudinal planes, an object is identified and reconstructed.

At stage 608, the elevation of the object is calculated. Referring to FIG. 17B, in general, the delay in the time an object appears in each of the beams depends on the elevation of the object. The delay between when an object appears in a particular view relative to its neighbor increases with elevation. Calculating the delay between when an object presents itself into each view implies a unique elevation. This calculation is done in each detector plane for each pair of first/last line and for each object under investigation. For example, object 601a is higher in the tunnel 12 than object 601b. The object at the higher elevation 601a is scanned for a longer time, and is seen earlier and later in the beam. It also intersects the fan beams at wider intervals.

At stage 610, the shape of the object is estimated based on a 12 sided polygon. The 12 sides are based on the leading and trailing edges of the 6 x-ray beams in the scanner 80 (i.e., 5 beams from the bottom source, and one form the side source). The 12 sided polygon is exemplary as a different polygon can be used based on the number of detection arrays in a scanner. Referring to FIGS. 17C and 17D, the 12 sided polygon can be used to find the boundary of the object under consideration. Some sides may have zero length if it happened to have sharp edges. For example, a rectangle 601c placed horizontally may have 4 real sides because the corners are intersected by 4 beams. The exact shape of the object may remain unknown but the 12 sided polygon is an upper bound for the area. In general, all that is known is that the true projection touches each of the sides of the object. Algorithms with estimations can be used to gap the missing points. For example, calculations as to the likelihood that the object under investigation is a circle 601d, ellipse, truncated ellipse (e.g., partially filled bottle), square, thin bulk or sheet, triangle, or other shapes. Each of the estimations can be assigned a confidence factor. Based on the calculations for the shape, the volume is determined as a function of the associated polygon, the confidence factor, and the area for each detector plane. At stage 612, the volume information is used to calculate the mass and density of the object.

At stage 614, the value of Zeff of the object is calculated. For each region, the elevation corrected background subtracted mass using both the high and low images is used. In an embodiment, an Alvarez-Macovski material decomposition scheme can be used to decompose the high and low images. The Zeff is calculated using the ratio of the high and low images. Alternatively, the Zeff can be determined by calculating each pixel's (or group of pixels') values and then averaging over the region.

Metal objects tend to have sharp edges and can be very obvious reference points. For example, wires can be seen in all views and their 3D location can be precisely determined, and then subtracted from the image to improve the Zeff calculations.

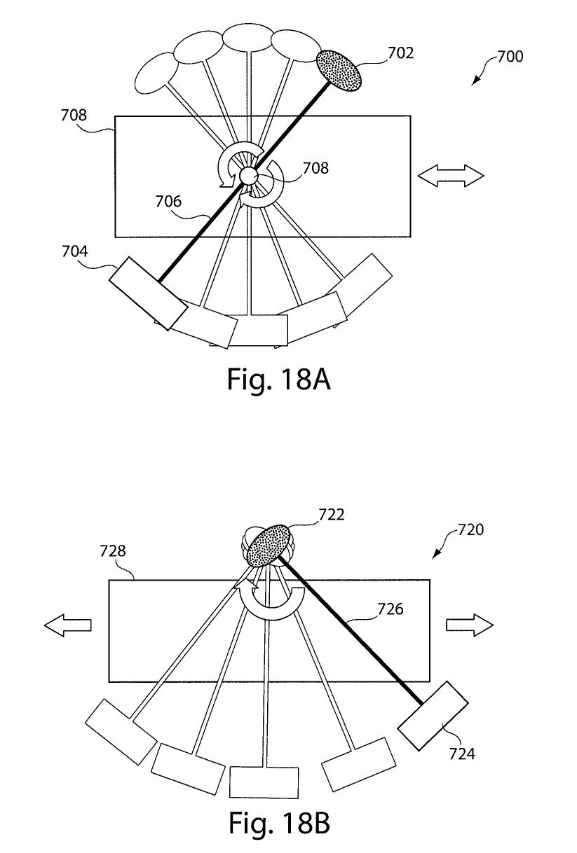

Referring to FIGS. 18A-B, block diagrams for container inspection scanners 700, 720. The scanners 700, 720 however, are exemplary only and not limiting. The scanners 700, 720 may be altered, e.g., by having components added, removed, or rearranged. The scanners 700, 720 include a high voltage x-ray source 702, 722 (e.g., Varian Linatron K15), a detector array 704, 724, and a pivot arm 706, 726. In an embodiment, referring to FIG. 18A, the scanner 700 includes centrally located pivot point 708. The high voltage source 702 is configured to produce an x-ray fan beam. The output power of the beam can vary based on application and object to be scanned. For example, a sea going shipping container may require a 9 MeV source. The detector array 704 is disposed on a pivot arm 706, and is configured to receive the x-ray fan beam. In operation, source 702 and detector 704 assembly is secured in a first position. A container 708 is then moved forward between sourced 702 and the detector 704. When the container has reached the extent of the first movement, the pivot arm 706 can be moved to a second position, and the container can be moved backwards between the source 702 and the detector 704. When the container 708 reaches it initial position again (i.e., it has completed its backwards movement), the pivot arm 706 can be rotated to a third position, and the container 708 is moved forward again. The process can continue through a number of rotational positions of the pivot arm 608. In an embodiment, a complete scan is obtained with five different positions of the source and detector assembly. The scan information can be processed by an image computer as describe above.

In an embodiment, referring to FIG. 18B, a scanner 720 includes a high voltage source 722, a detector array 724 and a pivot arm 726. The x-ray source 722 is disposed on or about a rotating surface such that source 722 is substantial near the axis of the source-detector assembly. For example, the pivot arm 726 and detector 724 can swing through an arc which is centered on the source 722. In operation, the pivot arm 726 can be located in a first position, and a container 728 can be moved between the source 722 and the detector 724 as described above. The relative movements of the source 702, 722, detector 704, 724, and container 708, 728 are exemplary only and not a limitation. Other movement and position combination can be used to obtain the image data.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

What is claimed is:

1. An x-ray scanning system, comprising:
a conveyor disposed at least partially in a tunnel and configured to move an object to be scanned through the tunnel along a direction of travel;
a first x-ray source disposed beneath the tunnel and configured to project a plurality of fan beams from a first focal point through the tunnel;
a first plurality of detector arrays, wherein each of the detector arrays is aligned to one of the fan beams projected from the first x-ray source;
a second x-ray source disposed on the side of the tunnel and configured to project a fan beam from second focal point through the tunnel; and
a second detector array aligned to the fan beam projected from the second x-ray source.

2. The x-ray scanning system of claim 1 wherein the second focal point is a height that is higher than the conveyor.

3. The x-ray scanning system of claim 2 wherein the height of the second focal point is approximately 8 inches above the conveyor.

4. The x-ray scanning system of claim 1 wherein the first x-ray source is configured to project five fan beams from the first focal point.

5. The x-ray scanning system of claim 4 wherein an angle between each of the fan beams is approximately 12.5 degrees.

6. The x-ray scanning system of claim 1 further comprising an image processing system configured to generate 3D images of a scanned object.

7. The x-ray scanning system of claim 6 further comprising an operator station with a display monitor and an input device, wherein an operator at the station can manipulate the input device to rotate the 3D images around a first pivot point.

8. The x-ray scanning system of claim 7 wherein the operator can manipulate the input device to rotate the 3D images around a second pivot point.

9. The x-ray scanning system of claim 1 wherein the first x-ray source is located in front of the second x-ray source along the direction of travel.

10. An array CT scanning system, comprising:
a tunnel;
a conveyor disposed at least partially within the tunnel can configured to move an object to be scanned through the tunnel along a direction of travel;
a single detector array disposed in proximity to the tunnel;
a plurality of x-ray sources disposed on the tunnel along the direction of travel, wherein each x-ray source is configured to project a fan beam towards the single detector; and
a control system operably coupled to the x-ray sources and configured to activate each of the x-ray sources sequentially such that only one x-ray source is projecting at a time.

11. The array CT scanning system of claim 10 wherein the plurality of x-ray sources are disposed under the tunnel and the fan beams are projected to the top and a side of the tunnel.

12. The array CT scanning system of claim 10 wherein the plurality of x-ray sources are disposed on the side of the tunnel and the fan beams are projected to a side, the top, and the bottom of the tunnel.

13. The array CT scanning system of claim 10 wherein the plurality of x-ray sources is a single source comprising nanotube technology and is configured to project a plurality of fan beams to the single detector.

14. The array CT scanning system of claim 10 wherein the single detector array comprises a plurality of detector elements, wherein each element includes a low energy detector, a high energy detector, and a curved filter material disposed between the low and high energy detectors.

15. The array CT scanning system of claim 14 wherein the curved filter material is disposed in the detector array such that each of the fan beams generated from each of the plurality of x-ray sources is substantially normal to the surface of the curved filter.

16. A passenger baggage screening system, comprising:
a multi-beam x-ray scanner with a conveyor;
an operator image display screen;
a side tray disposed adjacent to the conveyor such that a bag under inspection can be moved from the conveyor to the side tray by an operator; and
a bin return system.

17. The passenger baggage screening system of claim 16 further comprising an image processing system configured to store image information.

18. The passenger baggage screening system of claim 17 wherein the stored image information can be selected and displayed on the operator image display screen.

19. The passenger baggage screening system of claim 17 wherein the operator image display screen includes an input device.

20. The passenger baggage screening system of claim 17 wherein the image information comprises 3D images of passenger baggage, and an operator can manipulate the input device to display and rotate the 3D images around a selectable pivot point.
